(19) United States
(12) Patent Application Publication
(10) Pub. No.: US 2025/0000559 A1
(43) Pub. Date: Jan. 2, 2025

(54) ORTHOPEDIC SURGICAL INSTRUMENT

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Justin Taber, Honolulu, HI (US); Daniel Santos, Westborough, MA (US); Noah Christie, Laguna Beach, CA (US); Manouchehr Goharlaee, Encinitas, CA (US); Bhaktprakash Patel, Anaheim, CA (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/708,018

(22) PCT Filed: Oct. 7, 2022
(86) PCT No.: PCT/US22/46044

§ 371 Date: May 7, 2024

Related U.S. Application Data
(60) Provisional application No. 63/281,451, filed on Nov. 19, 2021.

(51) Int. Cl. A61B 17/92

(52) U.S. Cl. CPC A61B 17/92; A61B 2017/924

(57) ABSTRACT

An orthopedic surgical instrument or impactor arranged and configured to convert rotational motion of a motor into linear (e.g., reciprocating) motion of an internal hammer to drive an orthopedic implant (e.g., an acetabular cup, a femoral hip implant, an intramedullary nail, etc.) and/or a surgical tool (e.g., a broach, a rasp, a cutting tool, etc.) into a patient's bone. In addition, and/or alternatively, the orthopedic surgical instrument may be reversed to assist with removal of the orthopedical implant and/or surgical tool. In some examples, the orthopedic surgical instrument includes a swashplate and a wobble shaft to convert the rotational motion into linear motion. In addition, and/or alternatively, the orthopedic surgical instrument may be arranged and configured with multiple modes of operation to enable a user to selectively, and independently, adjust the impact energy, frequency, etc.

### CROSS-REFERENCE TO RELATED APPLICATIONS

This is a non-provisional of, and claims the benefit of the filing date of, pending U.S. provisional patent application No. 63/281,451, filed Nov. 19, 2021, entitled “Orthopedic Surgical Instrument,” the entirety of which application is incorporated by reference herein.

### TECHNICAL FIELD

The present disclosure is directed to an orthopedic surgical instrument, and more specifically to an orthopedic surgical instrument or impactor arranged and configured to transmit forward energy or motion to, for example, drive an orthopedic implant (e.g., an acetabular cup, an intramedullary nail, etc.) and/or a surgical tool (e.g., a broach) into a patient's bone, and reverse energy or motion to, for example, remove a stuck or lodged surgical tool (e.g., a broach) or implant from the patient's bone.

### BACKGROUND

Orthopedic surgical procedures such as, for example, hip procedures, knee procedures, shoulder procedures, etc., have become common place in today's society. For example, total hip arthroplasty or hip replacement is a well-known procedure for repairing damaged bone (e.g., a damaged hip). During a total hip arthroplasty, an acetabular system may be implanted into a patient's acetabulum. In addition, and/or alternatively, a femoral implant may be implanted into a patient's femur. During the surgical procedure, the patient's bone typically needs to be prepared to receive the orthopedic implant. For example, a surgical tool such as, for example, an orthopedic broach, rasp, cutting tool, etc. (terms used interchangeably herein without the intent to limit or distinguish) may be used to prepare an inner surface of a patient's intramedullary canal to receive an orthopedic implant such as, for example, a femoral hip prosthesis, an intramedullary nail, etc. The preparation of the intramedullary canal by the surgeon is designed to insure a proper fit between the patient's femur and the implant. In addition, the orthopedic implant such as, for example, the acetabular cup, may need to be impacted into proper position. Moreover, during removal of the broach from the patient's intramedullary canal, the broach may become stuck or lodged within the patient's intramedullary canal.

With this in mind, various surgical tools have been developed to assist surgeons during orthopaedic procedures to place and/or remove various objects. For example, mallets are frequently used to apply an impacting force on the orthopedic tool (e.g., broach) to remove bones or other implanted objects. In addition, the mallets may be used to assist in removing the orthopedic tool (e.g., broach) if it becomes stuck or lodged during the surgical procedure. Moreover, mallets may also be used to assist the surgeon with inserting the implant (e.g., mallets have been used to insert femoral nails, intramedullary nails, acetabular cups, etc.). While mallets are effective, the impacting force must be axially applied to avoid mishitting and/or misalignment of the implant or the inadvertent removal of the patient's bone. Moreover, the force applied should be sufficiently controlled to avoid unwanted damage to the patient's bone.

As a result, orthopedic impactors or slap hammers (terms used interchangeably herein without the intent to limit or distinguish) have been developed to assist with driving a surgical tool or implant into the patient's bone, and to remove a stuck or lodged surgical tool or implant from the patient's bone.

However, most orthopedic impactors still have several drawbacks. For example, orthopedic impactors may be large, heavy, and bulky, thus difficult to handle.

Accordingly, there remains a need for an improved orthopedic impactor. It is with this in mind that the present disclosure is provided.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein is an orthopedic surgical instrument or impactor arranged and configured to transmit a forward energy or motion to, for example, drive a surgical tool such as, for example, a broach, a chisel, a cutting tool, or other end effector known in the art, or an orthopedic implant such as, for example, an acetabular cup, an intramedullary nail, a femoral hip implant, etc. into a patient's bone. In addition, and/or alternatively, the orthopedic surgical instrument or impactor is arranged and configured to transmit a reverse energy or motion to, for example, remove a stuck or lodged surgical tool (e.g., a broach) or implant from the patient's bone.

In one example, the orthopedic surgical instrument or impactor includes a mechanical mechanism for converting rotary motion from a motor to linear motion. In various examples, the mechanical mechanism of the orthopedic surgical instrument or impactor includes a swashplate or swashplate mechanism and a wobble shaft arranged and configured to convert rotary motion from a motor (e.g., a DC motor) into reciprocating linear motion of an internal sliding hammer so that the hammer contacts, strikes, etc. an impact surface to drive the orthopedic implant or surgical tool into a patient's bone, or to remove the orthopedic implant or surgical tool from a patient's bone.

In any preceding or subsequent example, the orthopedic surgical instrument or impactor includes a housing arranged and configured to house or enclose one or more of the components of the orthopedic impactor, a rechargeable battery, a motor (e.g., a DC motor), and a trigger mechanism or assembly.

In any preceding or subsequent example, the motor is associated with an output shaft, which is coupled to the wobble shaft. The wobble shaft is also coupled to the swashplate. The swashplate is operatively coupled to the internal sliding hammer. In use, rotation of the motor rotates the output shaft, which rotates the wobble shaft. Rotation of the wobble shaft is converted into linear motion of the internal sliding hammer via interaction of the wobble shaft and the swashplate. Linear movement of the internal sliding hammer causes the internal sliding hammer to contact an impact or striking surface resulting in the driving (or removing) of the orthopedic implant and/or surgical tool.

In any preceding or subsequent example, the wobble shaft includes a first end and a second end, the first end operatively coupled to the output shaft of the motor, the second end operatively coupled to the swashplate. The second end of the wobble shaft being angled relative to the first end of the wobble shaft.

In any preceding or subsequent example, the first end of the wobble shaft includes an internal cavity arranged and configured to receive the output shaft of the motor.

In any preceding or subsequent example, the swashplate includes an internal bore arranged and configured to receive the second end of the wobble shaft.

In any preceding or subsequent example, an intermediate ball bearing assembly is disposed between an inner surface of the internal bore of the swashplate and an outer surface of the second end of the wobble shaft.

In any preceding or subsequent example, the swashplate includes a projection, leg, or the like extending therefrom, the leg being operatively coupled to the internal sliding hammer.

In any preceding or subsequent example, the internal sliding hammer is part of an internal hammer assembly including the internal sliding hammer, an impact mechanism housing, and a coupling mechanism such as, for example, a plunger and a pin.

In any preceding or subsequent example, the impact mechanism housing is operatively coupled to an end of the motor.

In any preceding or subsequent example, the impact mechanism housing includes a longitudinal slot formed in an outer surface thereof, the longitudinal slot arranged and configured to enable the leg of the swashplate to extend therethrough so that the leg can operatively engage the internal sliding hammer via the coupling mechanism (e.g., plunger and pin).

In any preceding or subsequent example, the orthopedic surgical instrument or impactor further includes a distal connector including a coupling mechanism for coupling, directly or indirectly, to an orthopedic implant or surgical tool. In some examples, the coupling mechanism is arranged and configured to selectively engage an adapter from a plurality of adapters. The adapters being arranged and configured to engage various surgical tools or orthopedic implants.

In any preceding or subsequent example, the coupling mechanism includes an internal cavity or opening formed in the distal connector, the internal cavity arranged and configured to receive a shaft of the adapter, surgical tool, or orthopedic implant. The coupling mechanism further including a pair of spring-loaded fingers arranged and configured to engage the shaft.

In addition, and/or alternatively, in any preceding or subsequent example, the orthopedic surgical instrument or impactor includes a control mechanism such as, for example, software, firmware, hardware, or a combination thereof, to alter, adjust, etc. the impact energy, frequency, or a combination thereof. For example, the orthopedic surgical instrument or impactor may include multiple modes of operation to enable a user to vary the impaction energy, frequency, or a combination thereof. In some examples, the orthopedic surgical instrument or impactor may include a full swing mode arranged and configured to provide maximum impact energy, a flutter mode arranged and configured to enable the hammer to oscillate with variable frequencies and/or amplitudes to provide increased frequency with reduced energy, an oscillation mode arranged and configured to operate the motor in one direction to impact both forward and reverse impact surfaces, or a combination thereof. Thus arranged, in some examples, the impact energy provided by the orthopedic impactor is adjustable.

Exemplary embodiments of the present disclosure provide numerous advantages. For example, the orthopedic surgical instrument enables a surgeon to accurately and safely apply a force to an orthopedic implant or a surgical tool. For example, the orthopedic surgical instrument may be arranged and configured to apply a force to a broach used to prepare an intramedullary canal of a patient's bone and/or to an orthopedic implant. The orthopedic surgical instrument is arranged and configured to apply a force to the broach and/or to the orthopedic implant, while minimizing the risk of injury to the patient or to the surgeon's hands during use. Moreover, in some examples, the orthopedic surgical instrument may be arranged and configured to enable the surgeon to separately and independently adjust the force applied by the orthopedic surgical instrument.

Further features and advantages of at least some of the exemplary embodiments of the present invention, as well as the structure and operation of various exemplary embodiments of the present invention, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 illustrates a perspective view of an example orthopedic surgical instrument or impactor in accordance with one or more features of the present disclosure;

FIG. 2 illustrates a side view of the orthopedic surgical instrument or impactor shown in FIG. 1, FIG. 2 shown with a transparent housing to illustrate some of the internal components of the orthopedic surgical instrument or impactor;

FIG. 3 illustrates an exploded perspective view of the orthopedic surgical instrument or impactor shown in FIG. 1;

FIG. 4A illustrates a top view of an example wobble shaft in accordance with one or more features of the present disclosure;

FIG. 4B illustrates a side view of the wobble shaft shown in FIG. 4A;

FIG. 4C illustrates a cross-sectional view of the wobble shaft shown in FIG. 4A, the cross-sectional view taken along line IVC-IVC in FIG. 4A;

FIG. 5 illustrates a view of an example swashplate in accordance with one or more features of the present disclosure;

FIG. 6 illustrates various views illustrating movement of the swashplate and internal hammer assembly during rotation of the motor in accordance with one or more features of the present disclosure;

FIG. 7A illustrates various views illustrating movement of the swashplate and internal hammer assembly during rotation of the motor in accordance with one or more features of the present disclosure, FIG. 7A illustrating impaction in the forward direction;

FIG. 7B illustrates various views illustrating movement of the swashplate and internal hammer assembly during rotation of the motor in accordance with one or more features of the present disclosure, FIG. 7B illustrating impaction in the reverse direction;

FIG. 8 illustrates an exploded perspective view of an example distal connector in accordance with one or more features of the present disclosure;

FIG. 9 illustrates a perspective view of an example adapter that can be coupled to the orthopedic surgical instrument or impactor in accordance with one or more features of the present disclosure;

FIG. 10A illustrates an example block diagram of an orthopedic surgical instrument or impactor in accordance with one or more features of the present disclosure;

FIG. 10B illustrates an example flowchart of the operation of the orthopedic surgical instrument or impactor in accordance with one or more features of the present disclosure;

FIG. 11 illustrates an example computing device communicatively coupled with the orthopedic surgical instrument or impactor in accordance with one or more features of the present disclosure;

FIGS. 12A-12C illustrate various examples of graphical displays of panels for a computing device to monitor and/or control an orthopedic surgical instrument or impactor in accordance with one or more features of the present disclosure;

FIGS. 13A-13C illustrate example flowcharts of motor operation in different modes of operation for an orthopedic surgical instrument or impactor in accordance with one or more features of the present disclosure;

FIG. 14 illustrates an alternate example computing device communicatively coupled with the orthopedic surgical instrument or impactor in accordance with one or more features of the present disclosure; and

FIGS. 15 and 16 illustrate example storage mediums and computing platforms for an orthopedic surgical instrument or impactor in accordance with one or more features of the present disclosure.

It should be understood that the drawings are not necessarily to scale and that the disclosed examples are sometimes illustrated diagrammatically and in partial views. In certain instances, details which are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

Various features or the like of an orthopedic surgical instrument or impactor (terms used interchangeably herein without the intent to limit or distinguish) arranged and configured to transmit a forward energy or motion to, for example, drive a surgical tool (e.g., a broach) or implant into a patient's bone, and a reverse energy or motion to, for example, remove a stuck or lodged surgical tool (e.g., a broach) or implant from a patient's bone will now be described more fully hereinafter with reference to the accompanying drawings, in which one or more features of the orthopedic impactor will be shown and described. It should be appreciated that the various features may be used independently of, or in combination, with each other. It will be appreciated that an orthopedic impactor as disclosed herein may be embodied in many different forms and should not be construed as being limited to the examples set forth herein. Rather, these examples are provided so that this disclosure will convey certain features of the orthopedic impactor to those skilled in the art.

As will be described herein, in accordance with one or more features of the present disclosure, the orthopedic impactor is arranged and configured to insert or implant an orthopedic implant such as, for example, an acetabular cup, an intramedullary nail, a femoral hip implant, etc. In addition, and/or alternatively, the orthopedic impactor may be coupled to a surgical tool such as, for example, a broach, to prepare a bone to receive an orthopedic implant. In some examples, the orthopedic impactor includes a swashplate or swashplate mechanism (terms used interchangeably herein without the intent to limit or distinguish) and a wobble shaft arranged and configured to convert rotational motion from a motor (e.g., a DC motor) into linear motion to drive an internal sliding hammer against an impact surface to drive (or remove) the orthopedic implant or tool into (or out) of a patient's bone.

In addition, and/or alternatively, in accordance with one or more features of the present disclosure, which may be used in combination with the swashplate and the wobble shaft, or separately therefrom, the orthopedic impactor may include multiple modes of operation to enable a user to vary the impaction energy, frequency, or a combination thereof. For example, the orthopedic impactor may include a full swing mode arranged and configured to provide maximum impact energy and a flutter mode arranged and configured to enable the internal sliding hammer to oscillate with variable frequencies and/or amplitudes to provide increased frequency with reduced energy.

In some examples, the orthopedic impactor is arranged and configured to provide a forward energy or motion to drive the orthopedic implant and/or surgical tool into a patient's bone. In addition, the orthopedic impactor may be reversed to provide a reverse energy or motion to, for example, remove a stuck or lodged surgical tool or implant from a patient's bone. In use, the user may select between the forward and reverse energy or motion by pushing forward or pulling back on the orthopedic impactor. That is, as will be described herein, in use, the user pushes forward on the orthopedic impactor causing the hammer to strike a first or forward impaction surface causing the orthopedic impactor to drive an orthopedic implant or surgical tool. Alternatively, in use, the user can pull back on the orthopedic impactor causing the hammer to strike a second or reverse impaction surface causing the orthopedic impactor to produce a reverse impaction to, for example, remove an orthopedic implant or surgical tool.

It should be appreciated that while, for example, the orthopedic impactor may be described herein in connection with driving a broach into a patient's bone to, for example, prepare an intramedullary canal of the patient's bone, the present disclosure is not so limited and the orthopedic impactor may be used in connection with any surgical tool or implant now known or hereafter developed. As such, the present disclosure should not be limited to any particular surgical tool, implant, or procedure unless explicitly claimed.

In accordance with one or more features of the present disclosure, the orthopedic impactor is arranged and configured to accurately and safely apply a force to an orthopedic implant or a surgical tool such as, for example, a broach to prepare an intramedullary canal of a patient's bone and/or to assist with removal of the broach from the intramedullary canal of the patient's bone. The orthopedic impactor is arranged and configured to apply a force, while minimizing the risk of injury to the patient or to the surgeon's hands during use. That is, in accordance with one or more features of the present disclosure, the orthopedic impactor helps the surgeon to deliver a force towards or away from a surgical area in a joint replacement procedure. For example, the orthopedic impactor is arranged and configured to provide a forward force to drive an orthopedic implant or surgical tool such as a broach to prepare an intramedullary canal of a patient's bone or to deliver a reverse force to assist a surgeon in removing a surgical implant or tool from a patient's bone.

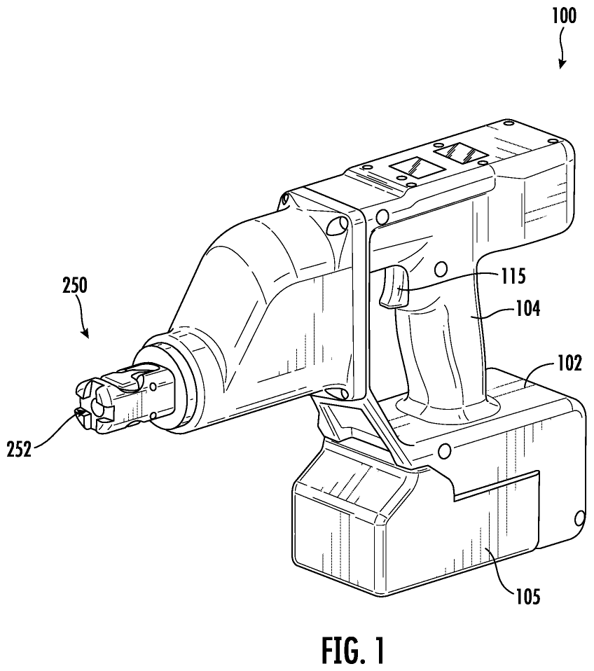

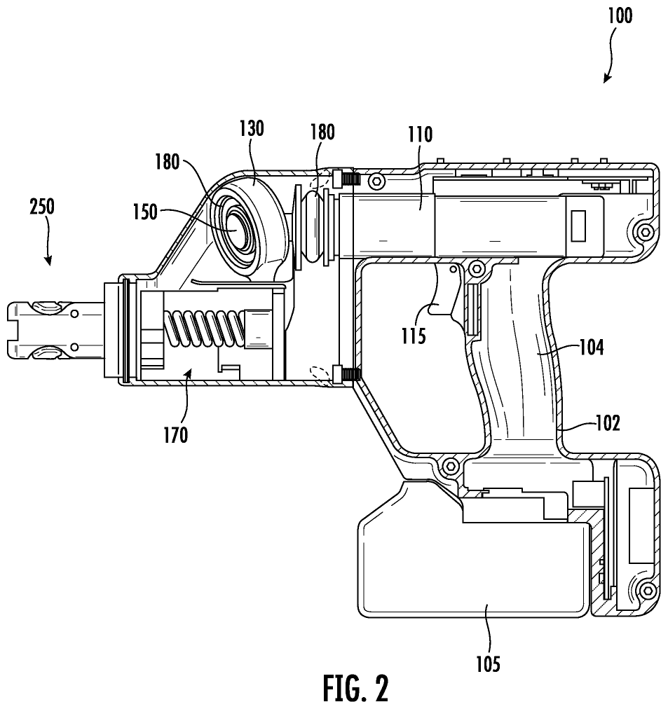

Referring to FIGS. 1-8, an example orthopedic impactor 100 will now be shown and described. As shown, the orthopedic impactor 100 may include a battery 105 so that the orthopedic impactor 100 is configured to be battery-powered as will be readily appreciated by one of ordinary skill in the art. Thus arranged, the orthopedic impactor 100 may provide wireless portability.

In use, the orthopedic impactor 100 includes a housing 102 arranged and configured to house or enclose one or more of the components of the orthopedic impactor 100. The housing 102 may be manufactured from any suitable material now known or hereafter developed such as, for example, a plastic or a metal. The housing 102 may also include a handle portion 104 with an optional hand grip for comfortable and secure holding of the orthopedic impactor 100. Alternatively, the housing 102 may incorporate a suitable mount interface for integrating the orthopedic impactor 100 into a robotic assembly while in use. In various examples, the housing 102 may be manufactured from multiple components that are assembled together.

The orthopedic impactor 100 may also include a motor 110 (e.g., a DC motor) and a trigger assembly 115. As will be readily appreciated by one of ordinary skill in the art, activation of the trigger assembly 115 (e.g., depressing the trigger) activates the motor 110 to rotate. As will be readily appreciated by one of ordinary skill in the art, incorporation of a trigger to activate the motor is well-known. Thus, for the sake of brevity, further discussion on operation and/or configuration of the trigger is omitted herefrom.

Thus arranged, the orthopedic impactor 100 is akin to current battery-powered screwdrivers, drills, etc. As such, for the sake of brevity of the present disclosure, further discussion on the operation and configuration of the battery, trigger, motor, etc. is omitted herefrom, such being readily understood by one of ordinary skill in the art. As will be appreciated however, the orthopedic impactor 100 may include any suitable motor and/or energy source (e.g., the orthopedic impactor 100 may be powered via AC power supplied through an electrical plug, etc.) now known or hereafter developed.

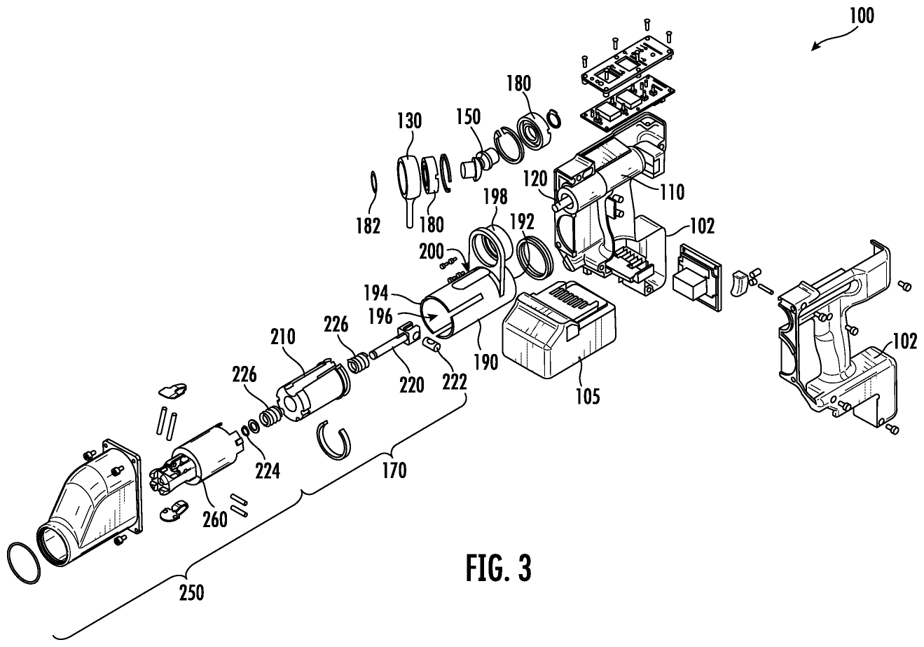

Referring to FIG. 3, in accordance with one or more features of the present disclosure, the motor 110 includes an output shaft 120. The orthopedic impactor 100 includes a swashplate 130, a wobble shaft 150, and an internal hammer assembly 170. In use, the wobble shaft 150 is operatively coupled to the output shaft 120 of the motor 110. In addition, the wobble shaft 150 is operatively coupled to the swashplate 130. The swashplate 130 is operatively coupled to the internal hammer assembly 170. As will be described in greater detail below, in use, rotation of the motor 110 rotates the output shaft 120, which rotation is transferred to the wobble shaft 150. Rotation of the wobble shaft 150 is converted into linear motion of the internal hammer assembly 170 via the interaction of the wobble shaft 150 and the swashplate 130. Linear movement of the internal hammer assembly 170 causes the internal hammer assembly 170 to contact a first or forward impact or striking surface 260 (terms used interchangeably without the intent to limit or distinguish) resulting in the driving of the orthopedic implant and/or surgical tool, or a second or reverse impact or striking surface 262 (terms used interchangeably without the intent to limit or distinguish) resulting in a reverse energy or motion.

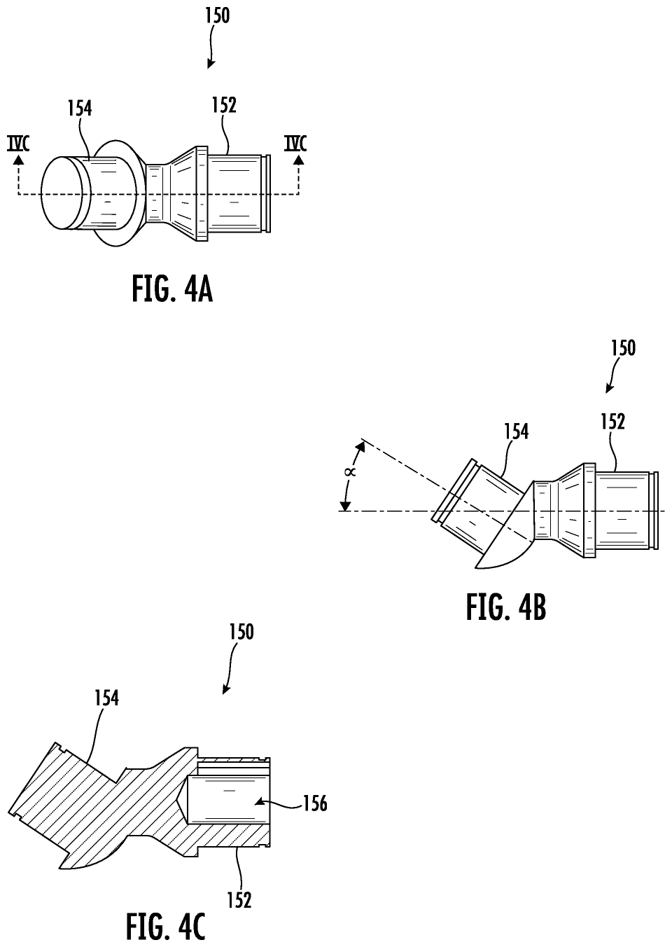

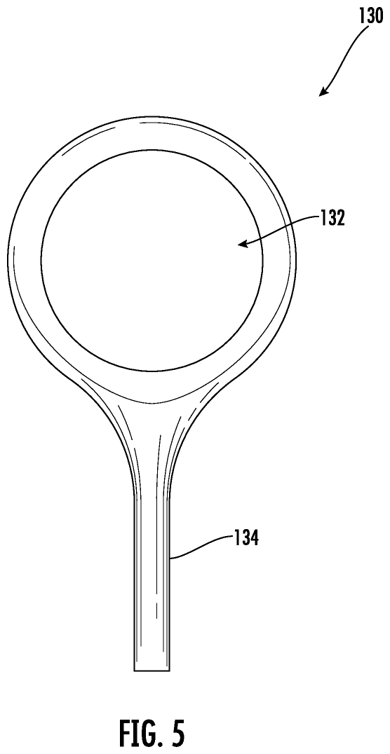

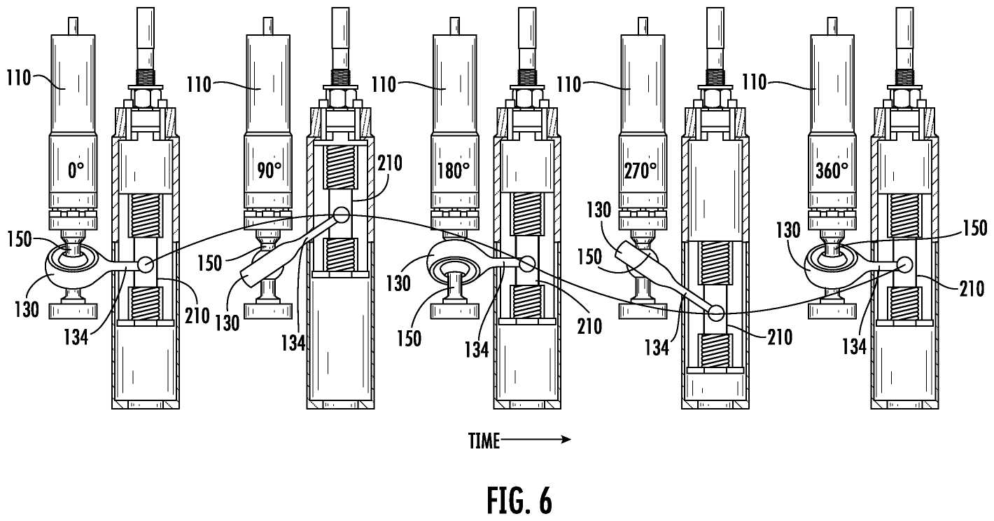

Referring to FIGS. 4A-4C, the wobble shaft 150 includes a first end 152 and a second end 154. The first end 152 includes an internal cavity 156 arranged and configured to receive the output shaft 120 of the motor 110, although this is but one configuration and the wobble shaft 150 may be coupled to the output shaft 120 of the motor 110 via any other suitable mechanism now known or hereafter developed. Thus arranged, rotation of the output shaft 120 rotates the wobble shaft 150. The second end 154 of the wobble shaft 150 is operatively coupled to the swashplate 130. As illustrated, the swashplate 130 may include an internal bore 132 arranged and configured to receive the second end 154 of the wobble shaft 150. In some examples, an intermediate ball bearing assembly 180 (FIG. 3) may be disposed between the surface of the internal bore 132 of the swashplate 130 and the outer surface of the second end 154 of the wobble shaft 150. In addition, a retaining ring 182 (FIG. 3) may be used to couple the swashplate 130 to the second end 154 of the wobble shaft 150, although this is but one configuration and the swashplate 130 may be coupled to the wobble shaft 150 via any other suitable mechanism now known or hereafter developed.

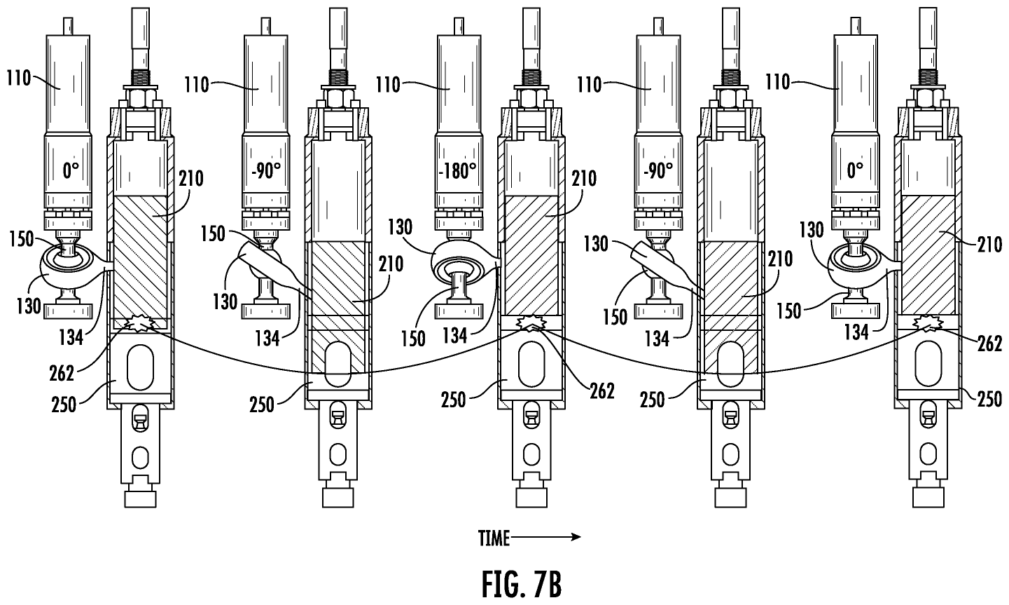

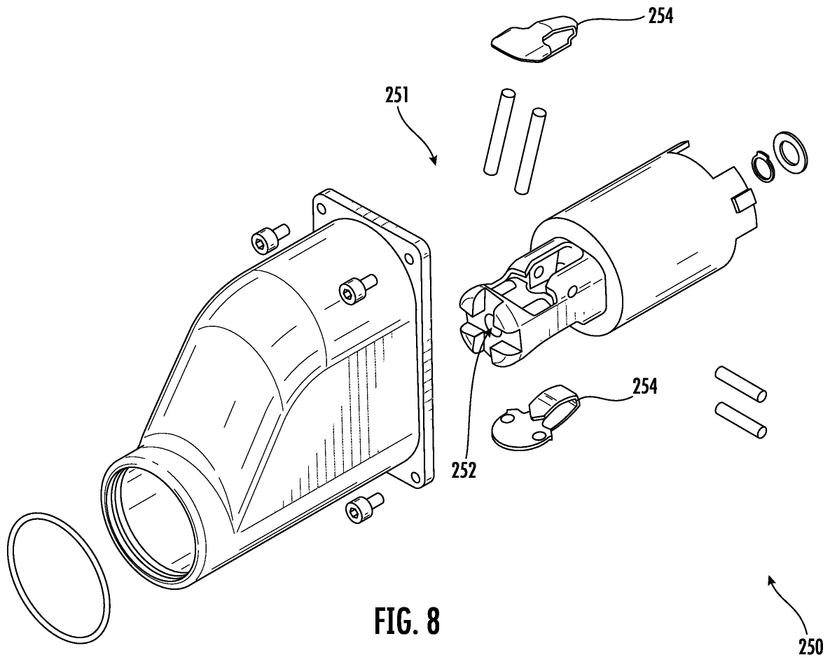

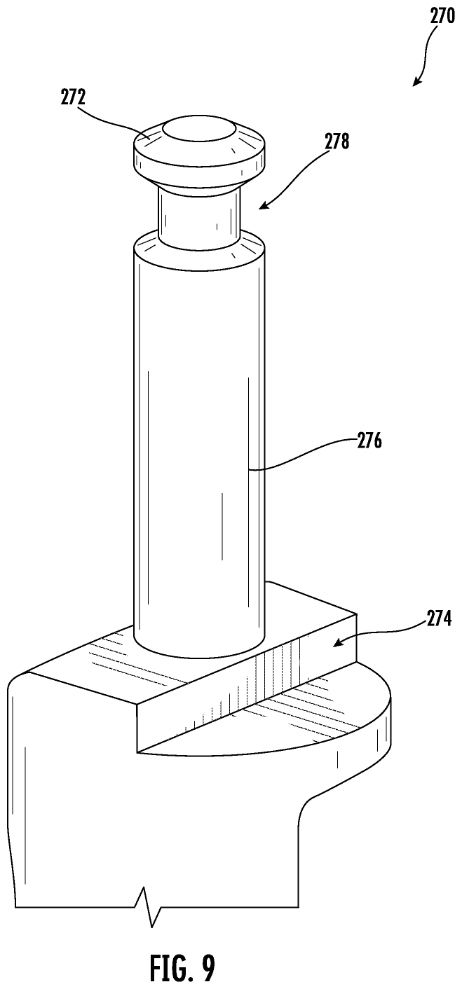

As illustrated in FIGS. 4A-4C, the second end 154 of the wobble shaft 150 is angled relative to the first end 152 of the wobble shaft 150. Thus arranged, as will be described in greater detail below and as generally illustrated in FIGS. 6, 7A, and 7B, rotation of the first end 152 of the wobble shaft 150 via rotation of the motor 110 causes the second end 154 of the wobble shaft 150 to rotate, which causes the swashplate 130 to convert the rotational motion of the wobble shaft 150 into linear motion. That is, as will be appreciated by one of ordinary skill in the art, the second end 154 of the wobble shaft 150 defines an inner race for the intermediate ball bearing assembly 180 and the swashplate 130. Rotation of the wobble shaft 150 (e.g., inner raceway) changes the angle of rotation, which gets converted into a reciprocating linear motion based on the position of the motor 110 (e.g., angular position of the motor 110 and output shaft 120). That is, by angling the second end 154 of the wobble shaft 150 relative to the first end 152 of the wobble shaft 150, the angle of rotation of the swashplate 130 is angled relative to the longitudinal axis of rotation (e.g., longitudinal axis of the output shaft 120 of the motor 110). In addition, the swashplate 130 is prevented from rotating. Thus arranged, rotation of the motor 110 changes the angle of the rotation of the ball bearing assembly 180, which causes a reciprocating linear motion of the leg 134 of the swashplate 130. In some examples, the second end 154 of the wobble shaft 150 may be angled relative to the first end 152 of the wobble shaft 150 by an angle α. In some examples, angle α may be between approximately 27 degrees to 37 degrees, preferably approximately 32 degrees.

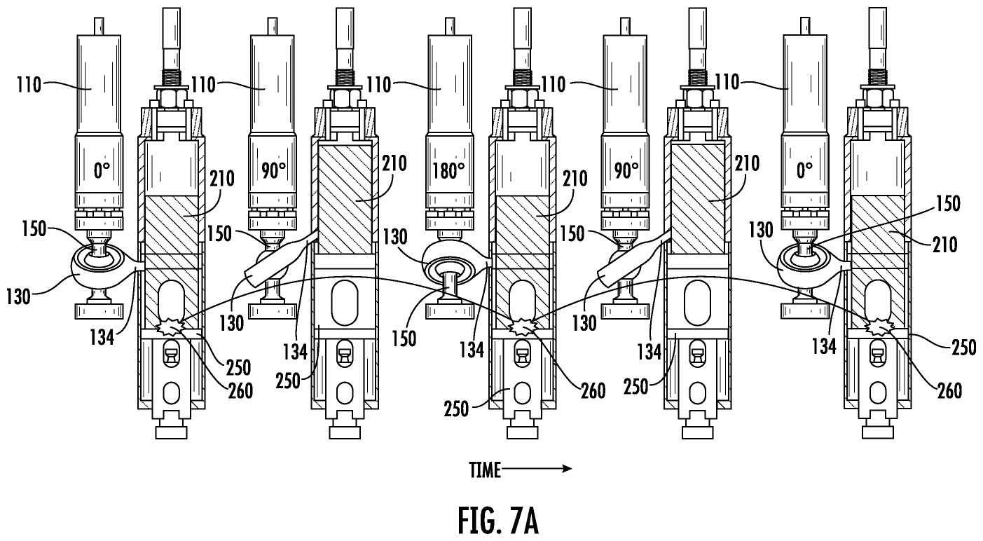

Referring to FIG. 5, in some examples, the swashplate 130 includes a projection, leg, or the like extending therefrom. In use, the leg 134 is operatively coupled to the internal hammer assembly 170 such that rotation of the wobble shaft 150 via the motor 110 causes movement of the swashplate 130, which linearly moves the internal hammer assembly 170 back and forth.

As illustrated in FIG. 3, the internal hammer assembly 170 may include an impact mechanism housing 190, a hammer 210, and a coupling mechanism (e.g., a plunger 220, a pin 222, and a retaining ring 224). In use, the plunger 220, the pin 222, and the retaining ring 224 are arranged and configured to couple the swashplate 130 to the hammer 210 so that rotation of the motor 110 and the wobble shaft 150 moves, pivots, oscillates, etc. the swashplate 130 causing the hammer 210 to linearly move. The internal hammer assembly 170 may also include a pair of springs 226 such as, for example, coiled springs. In use, the springs 226 may be positioned on either side of the hammer 210 to bias the hammer 210. In use, the springs 226 are arranged and configured to absorb shock and shield the motor 110 and swashplate 130 from any sudden deacceleration of the hammer 210 upon impact in either the forward or reverse direction.

The impact mechanism housing 190 may include a first end 192, a second end 194 and an internal bore 196 extending between the first and second ends 192, 194. The hammer 210, the plunger 220, and the pin 222 may be positioned within the internal bore 196 of the impact mechanism housing 190. In use, as illustrated, the impact mechanism housing 190 may be coupled to the end of the motor 110. For example, as illustrated, the impact mechanism housing 190 may include an arm 198 having a bore extending therefrom. In use, the output shaft 120 may pass through the bore formed in the arm 198 of the impact mechanism housing 190. An intermediate ball bearing assembly 180 may be positioned between the output shaft 120 and the inner surface of the bore of the impact mechanism housing 190. In addition, the impact mechanism housing 190 may include a longitudinal slot 200 formed in an outer surface thereof. In use, the leg 134 of the swashplate 130 may extend through the longitudinal slot 200 formed in the outer surface of the impact mechanism housing 190. Thus arranged, the leg 134 may engage the hammer 210 positioned within the internal bore 196 of the impact mechanism housing 190. In some examples, the leg 134 may be coupled to the hammer 210 via the plunger 220 and the pin 222, although this is but one configuration and the leg 134 of the swashplate 130 may be coupled to the hammer 210 via any other suitable mechanism now known or hereafter developed.

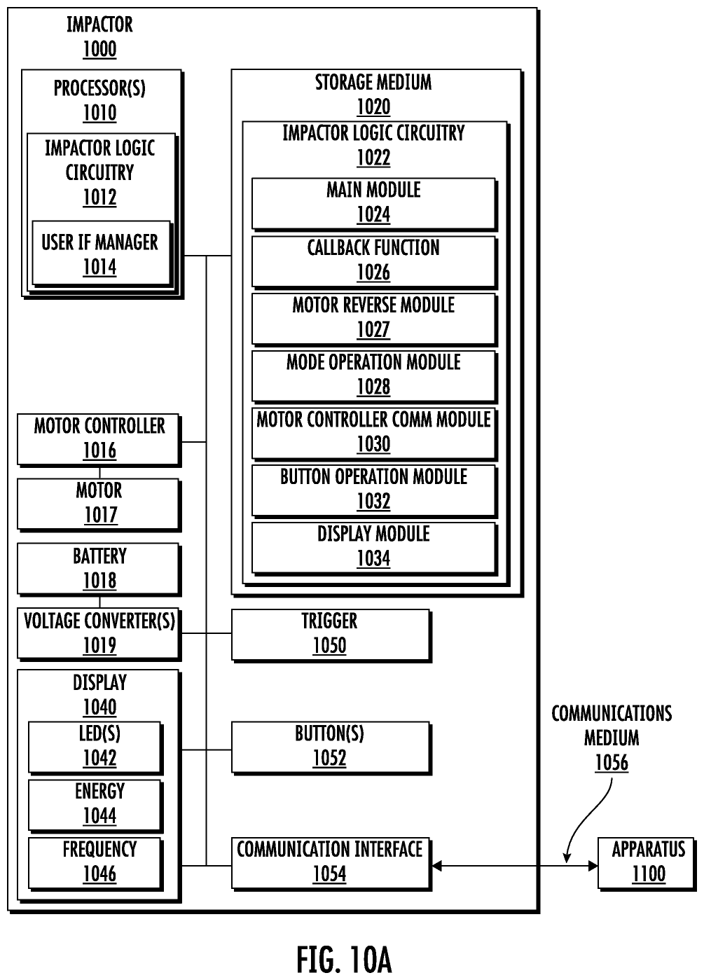

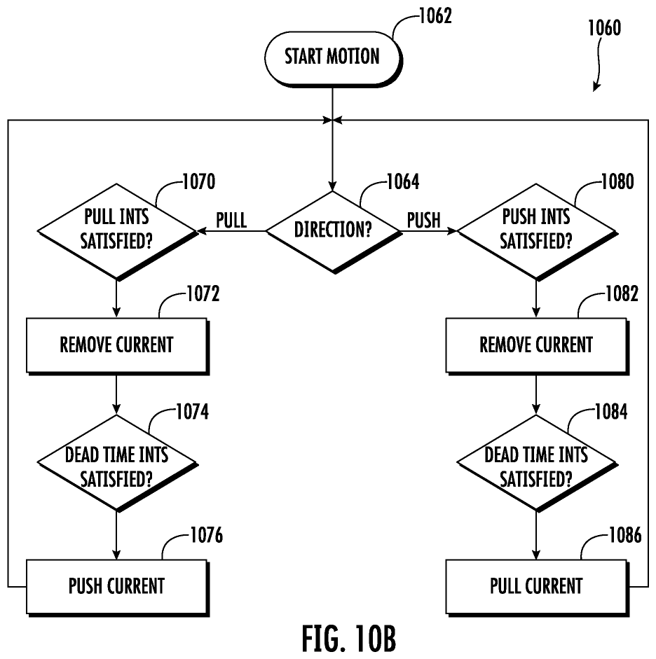

Referring to FIGS. 3 and 8, the orthopedic impactor 100 may also include a distal connector 250. In use, the distal connector 250 includes a coupling mechanism 251 for coupling to an orthopedic implant or surgical tool such as, for example, a broach, a chisel, a cutting tool, or other end effector known in the art. The coupling mechanism 251 may have a quick connect mechanism to facilitate rapid change. In some examples, the coupling mechanism 251 is arranged and configured to selectively couple to an adapter, which is arranged and configured to couple to the orthopedic implant and/or surgical tool. For example, referring to FIG. 9, the distal connector 250 is arranged and configured to couple to an adapter 270. The adapter 270 includes a first end 272 and a second end 274. In use, the second end 274 is arranged and configured to engage one of an orthopedic implant and/or a surgical tool. The first end 272 is arranged and configured to engage the distal connector 250. In use, the distal connector 250 is arranged and configured to selectively couple to the adapter 270 so that the adapter 270 can be interchangeably. As such, any number of different adapters either currently manufactured or hereafter developed can be used. As such, the adapter 270 can be any suitable adapter now known or hereafter developed. An example of an adapter 270 is illustrated in FIG. 9. As illustrated, in some examples, the adapter 270 may include a shaft 276 having a groove 278 formed therein. The shaft 276 and groove 278 being receivable within the distal connector 250 of the orthopedic impactor 100. For example, in some examples, the distal connector 250 may include an internal cavity or opening 252 (FIG. 8) arranged and configured to receive a portion of the shaft 276 therein. The distal connector 250 may also include a pair of spring-loaded or spring-biased fingers 254 arranged and configured to engage the shaft 276. In some examples, the shaft 276 may be arranged and configured to be non-rotatably coupled to the distal connector 250. For example, the shaft 276 and/or internal cavity or opening 252 may include one or more flat sides arranged and configured to key the shaft 276 to the distal connector 250 to prevent relative rotation, however, this is but one configuration and any number of shafts, keys, etc. may be used.

In use, rotation movement of the output shaft 120 via activation of the motor 110 is transferred to the wobble shaft 150, which rotates relative to the swashplate 130, which converts the rotational motion into linear motion of the hammer 210. With reference to FIG. 7A, in use, by pushing forward on the orthopedic impactor 100, linear movement of the hammer 210 causes the hammer 210 to strike a distal end or a forward impact surface 260 of the distal connector 250, which transmits a forward energy or motion to an orthopedic surgical implant or surgical tool coupled to the orthopedic impactor 100. Thus arranged, rotational motion of the motor 110 is converted into linear motion of the hammer 210, which drives either the orthopedic implant and/or the surgical tool coupled thereto via, for example, an adaptor. Alternatively, with reference to FIG. 7B, in use, by pulling back on the orthopedic impactor 100, linear movement of the hammer 210 causes the hammer 210 to strike a proximal end or a reverse impact surface 262 of the distal connector 250, which transmits a reverse energy or motion to an orthopedic surgical implant or surgical tool coupled to the orthopedic impactor 100. Thus arranged, rotation motion of the motor 110 is converted into linear motion of the hammer 210, which drives either the orthopedic implant and/or the surgical tool coupled thereto via, for example, an adapter. By pushing forward or pulling back on the orthopedic impactor 100, the relative position of the hammer 210 is changed relative to the distal connector 250. As such, by either pushing forward or reverse, the hammer 210 contacts either the forward impact surface 260 or the reverse impact surface 262.

In addition, and/or alternatively, in accordance with one or more features of the present disclosure, which may be used in combination with the swashplate and wobble shaft, or separately therefrom, the orthopedic impactor 100 may include multiple modes of operation to enable a user to vary the impaction energy, frequency, or a combination thereof. For example, the orthopedic impactor 100 may include a full swing mode arranged and configured to provide maximum impact energy, a flutter mode arranged and configured to enable the hammer to oscillate with variable frequencies and/or amplitudes to provide increased frequency with reduced energy; and an oscillation mode arranged and configured to enable the hammer to oscillate between the forward impact surface 260 and the reverse impact surface 262 of the distal connector 250 to provide increased frequency with reduced energy. Thus arranged, in some examples, the impact energy provided by the orthopedic impactor is adjustable.

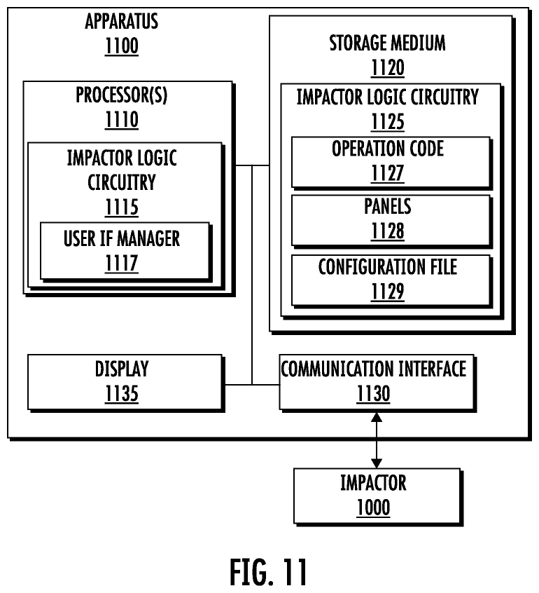

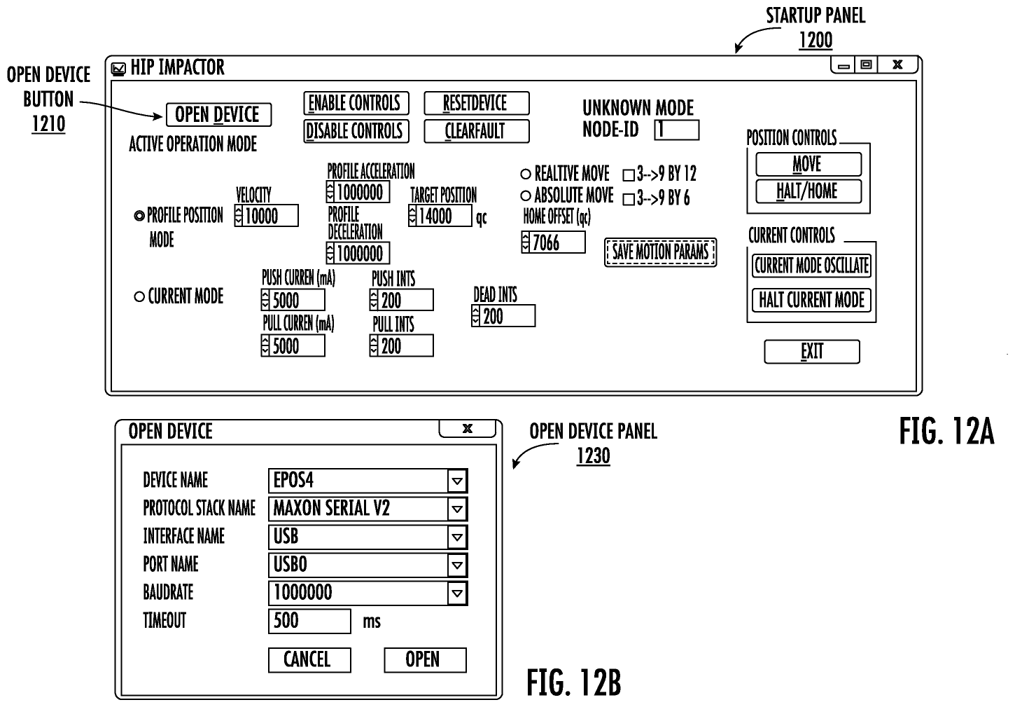

FIG. 10A illustrates an example block diagram of an orthopedic surgical instrument or impactor in accordance with one or more features of the present disclosure. FIG. 10B illustrates an example flowchart of the operation of the orthopedic impactor 1000 in accordance with one or more features of the present disclosure.

FIG. 10A depicts an example block diagram of the operation of an orthopedic impactor 1000 such as, for example, orthopedic impactor 100 shown in connection with FIGS. 1-8. The orthopedic impactor 1000 can be combined with any suitable example system, device, and method disclosed herein. The orthopedic impactor 1000 can include processor(s) 1010, a non-transitory storage medium 1020, a motor controller 1016, a motor 1017, a battery 1018, voltage converter(s) 1019, a display 1140, a trigger 1050, button(s) 1052, and a communication interface 1054. The processor(s) 1010 may include one or more processors, such as a programmable processor, a micro-controller unit (MCU), an application specific integrated circuit (ASIC), a system-on-a-chip (SOC), and/or the like. The processor(s) 1010 may include processing circuitry to implement impactor logic circuitry 1012 and 1022.

The processor(s) 1010 may operatively couple with a non-transitory storage medium 1020. The non-transitory storage medium 1020 may store logic, code, and/or program instructions executable by the processor(s) 1010 for performing one or more operations including the operations of the impactor logic circuitry 1022. The non-transitory storage medium 1020 may include one or more memory units (e.g., fixed or removable media or external storage such as a flash memory, secure digital (SD) card, random-access memory (RAM), read only memory (ROM), a flash drive, a hard drive, a solid-state drive (SSD) and/or the like). The memory units of the non-transitory storage medium 1020 can store logic, code, and/or program instructions executable by the processor(s) 1010 to perform any suitable example method described herein. For example, the processor(s) 1010 may execute instructions such as instructions of impactor logic circuitry 1125 causing the motor 1017 to rotate a shaft to operate a hammer such as the hammer 210 in FIG. 3 to impact a forward impact surface 260 of a distal connector 250 and/or impact a reverse impact surface 262 of a distal connector 250 at an impact energy and frequency selected by a user via button(s) 1052 and/or via apparatus 1100 (e.g., a computing device that may be communicatively coupled with the orthopedic impactor as will be described in greater detail below).

The processor(s) 1010 may include code for the orthopedic impactor 1000 in memory within the processor(s) 1010 and/or closely connected to the processor(s) 1010 such as flash memory. The impactor logic circuitry 1012 may represent code in or near the processor(s) 1010 for execution by the processor(s) 1010 and may include a user interface manager 1014. The user interface manager 1014 may include code executing on the processor(s) 1010 to detect and respond to user input as well as to detect the motor controller 1016 (such as a Maxon EPOS4 Controller) and establish communication with the motor controller 1016.

The user interface manager 1014 may communicate with the motor controller 1016 to receive status information about the motor 1017 and to control operation of the motor 1017. For instance, all button presses of button(s) 1052 and edit events may be posted to the user interface manager 1014 and processed in real-time. The user interface manager 1014 may communicate commands with the motor controller 1016 to execute in response to the user's actions via button presses, system states, and error conditions. The user interface manager 1014 may communicate alerts, warnings, and notifications to a user via the display 1040 and or the apparatus 1100 via the communications interface 1054. Furthermore, the user interface manager 1014 may also handle the user's response to alerts.

The motor 1017 may include a DC motor such as the motor 110 discussed in conjunction with FIG. 3. Similarly, the battery 1018 may include a power source for the orthopedic impactor 1000 components shown in FIG. 10A such as the battery 105 shown in FIG. 3.

The voltage converter(s) 1019 may include a DC-DC voltage converters to adjust the voltage of signals to various voltages required to operate the components of the orthopedic impactor 1000 such as the processor(s) 1010, the non-transitory storage medium 1020, the motor controller 1016, the display 1040, the trigger 1050, the button(s) 1052, the communications interface 1054, and/or the like.

The non-transitory storage medium 1020 may include code for execution by the processor(s) 1010 to operate the orthopedic impactor 1000. As needed, the processor(s) 1010 may copy code from the non-transitory storage medium 1020 to memory closer to the processor(s) 1010 to facilitate faster execution of the code. For instance, the user interface manager 1014 may include code copied from the impactor logic circuitry 1022 to memory closer to the processor(s) 1010 for execution.

The impactor logic circuitry 1022 may include code for operation of the orthopedic impactor 1000 stored in hardware of the storage medium such as volatile or non-volatile memory in the non-transitory storage medium 1020. The impactor logic circuitry 1022 may include a main module 1024, a callback module or function 1026, a motor reverse module 1027, a mode operation module 1028, a motor controller communications module 1030, a button operation module 1032, and a display module 1034.

The main module 1024 may include setup and loop functions. The setup function may run once at start-up and the loop function may run continuously afterwards. The setup function may attach interrupts that run when button(s) 1052 are pressed on the user interface, initializes Timer1 which runs the trigger interrupt service routine (ISR), and initializes an impact delay for the motor 1017. The loop function allows the motor 1017 may operate in the user-desired mode when the trigger 1050 is enabled and pulled. The loop function also handles showing the user that the trigger state is enabled via LED(S) 1042 of the display 1040 and/or via the apparatus 1100.

The callback function 1026 may be, e.g., an ISR that runs every millisecond. In other examples, the callback function 1026 may run periodically with a time interval of more than one millisecond or less than one millisecond.

The motor reverse module 1027 may include functions to prepare to reverse the motor 1017, change a motor direction of the motor 1017, calculate impact delay of the hammer 210, and setup flutter time delays to set the frequency of impact while in flutter mode. These functions may switch the direction of the motor 1017, reversing the motor 1017 to allow for bi-directional operation of the hammer 210, and may also determine the delay between reversals for controlling the frequency of impacts of the hammer 210 in flutter mode.

The mode operation module 1028 may include the functions of position check, flutter check, and oscillation check that are called for in normal/full-swing mode, high-frequency/flutter mode, and oscillation mode respectively. Normal mode operation checks the position of the motor 1017 then calls the prepare to reverse function. In many examples, the position of the motor 1017 may be monitored via an encoder on a shaft of motor 1017 that produces a count responsive to increments of rotation of the stator or shaft of the motor 1017.

High-Frequency mode operation uses the delay determined for flutter mode to rapidly switch the direction of the motor 1017. In flutter mode, the hammer 210 may only impact the forward impact surface 260 of the distal connector 250 or may only impact the reverse impact surface 262 of the distal connector 250. A delay may be determined to apply at the time of reversal of the motor 1017 to adjust the frequency of impacts of the hammer 210.

Oscillation mode operation operates the motor 1017 in one direction, i.e., forward or reverse, for impacting both the forward impact surface 260 and the reverse impact surface 262 of the distal connector 250. Some examples may not include an oscillation mode. Some examples may include two modes from a group of modes including the full-swing mode, the flutter mode, and the oscillation mode.

The motor controller communication module 1030 may include the functions to enable the motor controller 1016 functions, set motor amperage (upper bound amperage), zero motor amperage (lower bound amperage), and disable the motor controller 1016 functions. These functions instruct the motor controller 1016 whether or not to operate the motor 1017 and set the operating amperage bounds for operation of the motor 1017.

The button operation module 1032 may include functions to handle setting the user-desired amperage and frequency to operate the motor 1017 in addition to setting the operation mode and enabling the trigger 1050. The functions include energy plus to increase the energy of impact by the hammer 210, energy minus to increase the energy of impact by the hammer 210, frequency plus to increase the frequency of impacts by the hammer 210, frequency minus to decrease the frequency of impacts by the hammer 210, select operating mode to switch between available modes of operation (e.g., full-swing mode, flutter mode, or oscillation mode), and set trigger state to enable or disable the trigger 1050. In many examples, these functions may be accessed via the apparatus 1100 and/or the button(s) 1052. In other examples, a touch screen may be included in the display in lieu of or in addition to the button(s) 1052.

The display module 1034 may include functions handle the logic for displaying the amperage and frequency on the user interface. The functions may include an energy display and a frequency display.

The display 1040 may include LED(s) and numerical, alphanumeric, or graphical displays such as LED displays or liquid crystal displays (LCDs) to present a number representative of the energy 1044 and the frequency 1046 selected for operation of the motor 1017. The button(s) 1052 may include one or more buttons located in the display 1040 and, in some examples, may be located adjacent to the energy 1044 and frequency 1046 displays to provide a user with an interface to increase and/or decrease the energy and/or the frequency of the impact of the hammer 210 on the forward impact surface 260 and/or the reverse impact surface 262 of the distal connector 250.

The trigger 1050 may include a trigger or other button or switch that, when actuated, can cause the orthopedic impactor 1000 to operate if the trigger 1050 is enabled. If the trigger 1050 is disabled, depressing the trigger 1050 may not cause the orthopedic impactor 1000 to operate. In some examples, the trigger 1050 cannot be depressed when the trigger 1050 is disabled.

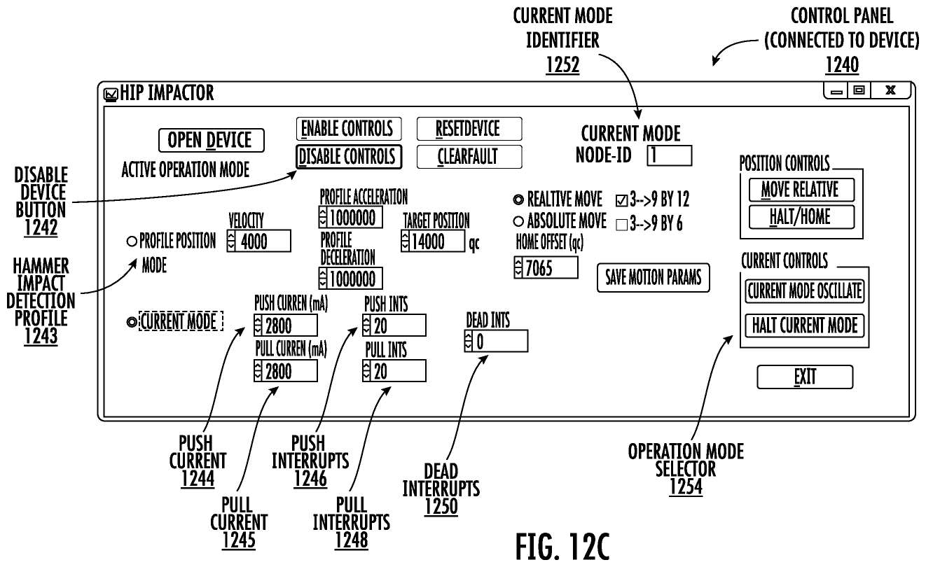

The processor(s) 1110 may couple to a communication interface 1054 to communicate with an apparatus 1100 via a communications medium 1056. The communications medium 1056 may include a wired or wireless interface to communicatively couple the orthopedic impactor 1000 with the apparatus 1100 shown in FIG. 11.

The communication interface 1054 may communicate user commands to and/or from the apparatus 1100 to the orthopedic impactor 1000 to operate the orthopedic impactor 1000 via the functionality described in conjunction with the orthopedic impactor 1000. In some examples, the apparatus 1100 may operate the motor 1017 in addition to configuring parameters of operation of the motor 1017 such as the upper current bound, the lower current bound, the operating current, the upper frequency bound, the lower frequency bound, the operating frequency, the mode of operation of the motor 1017, and/or the like. In some examples, the communication interface 1054 may communicate information about the operation of the orthopedic impactor 1000 to the apparatus 1100 such as the energy of operation, the frequency of operation, the mode of operation, events or alerts associated with the orthopedic impactor 1000, and log information such as time and date of use, impact detections, encoder counts, and/or the like. Note that the encoders may sense and provide feedback related to the position, count, speed, and/or direction of rotation of the motor 1017.

The communication interface 1054 includes circuitry to transmit and receive communications through a wired and/or wireless media such as an Ethernet interface, a wireless fidelity (Wi-Fi) interface, a cellular data interface, and/or the like. In some examples, the communication interface 1054 may implement logic such as code in a baseband processor to interact with a physical layer device to transmit and receive wireless communications to/from apparatus 1100. For example, the communication interface 1130 may implement one or more of local area networks (LAN), wide area networks (WAN), infrared, radio, Wi-Fi, point-to-point (P2P) networks, telecommunication networks, cloud communication, and the like.

FIG. 10B depicts an example flowchart 1060 for operation of a motor of an impactor such as the motor 1017 of the orthopedic impactor 1000 shown in FIG. 10A. The flowchart 1060 begins with starting motion of the motor (element 1062). The motion may be started in any direction 1064, either pulling the hammer or pushing the hammer such as the hammer 210 shown in FIGS. 7A and 7B.

In the full swing mode, the hammer may be pulled until an impact is detected. The impact includes an impact on the reverse impact surface 162 and the impactor logic circuitry may advantageously detect the impact via a sharp deceleration of the hammer as evidenced by a change in the speed or quantity of counts received from the motor 1017 encoder. In some examples, the impactor logic circuitry may monitor for a reduction in the count below a threshold or by a threshold deceleration of the counts. In some examples, the counts may vary based on a gear ration of the gear box coupled with the motor 1017. The gear ratio may affect the granularity of the stator movement of the motor 1017 per count, reducing the number of counts per stator rotation for gear boxes with low gear ratios such as 4.8:1 as compared with the number of counts per stator rotation for gear boxes with higher gear ratios such as 14:1. In such examples, a threshold count may be different depending on the gear ratio of the gear box connected to the motor 1017.

After impact, the impactor logic circuitry may reverse the motor 1017 (the direction of rotation of the stator of the motor 1017). Before applying the reverse current, the impactor logic circuitry may determine if the number of interrupts received during pulling the hammer represent the selected number of interrupts (element 1070). In many examples, the movement of the motor is closely coupled with the movement of the hammer. The number of interrupts may represent the counts from the encoder of the motor or may represent counts of clock cycles so the impactor logic circuitry may determine whether the counts received at impact are within an expected range of counts for impact of the hammer 210 on the reverse impact surface 262 of the distal connector 250. If the counts differ from the expected range of the counts, an event and/or alert may be generated to indicate a potential problem with the orthopedic impactor 1000.

If the number of interrupts are satisfied, the impactor logic circuitry may remove current from the motor 1017 (element 1072) for a delay time (or dead time) (element 1074) that adjusts the frequency of impact of the hammer to a user selected frequency. The interrupts may represent clock cycles of a clock or may represent counts of stator movement determined to represent the amount of delay to include at the time of reversal of the motor 1017 to set the frequency of impacts at the frequency selected by the user. In some examples, the interrupts may represent the callback function such as the callback function 1026 shown in FIG. 10A and may, e.g., have a period of 1 millisecond. In other examples, the period may be less than one millisecond or more than one millisecond.

Once the delay time is satisfied, the impactor logic circuitry may apply a push current to the motor 1017 (element 1076) to rotate the stator of the motor and the shaft of the motor 1017 in the opposite direction to push the hammer forward towards the forward impact surface 260 of the distal connector. For example, in some examples, a 9-ampere current may be a low energy setting and a 20-ampere current may be a high energy setting.

After applying the push current, the flowchart returns to element 1080 via the direction 1064. At element 1080, the impact logic circuitry may determine if the number of interrupts received at impact are within an expected range of counts for impact of the hammer 210 on the forward impact surface 260 of the distal connector 250. If the counts differ from the expected range of the counts, an event and/or alert may be generated to indicate a potential problem with the orthopedic impactor 1000.

If the number of push interrupts are satisfied, the impactor logic circuitry may remove current from the motor 1017 (element 1082) for a delay time (or dead time) (element 1084) to adjust the frequency of impact of the hammer on the forward impact surface based on a user selected frequency. The interrupts may represent clock cycles of a clock or may represent counts of stator movement determined to represent the amount of delay time required at the time of reversal of the motor 1017 to set the frequency of impacts at the frequency selected by the user. In some examples, the interrupts may represent the callback function such as the callback function 1026 shown in FIG. 10A and may, e.g., have a period of 1 millisecond. In other examples, the period may be less than one millisecond or more than one millisecond.

Once the delay time is satisfied, the impactor logic circuitry may apply a pull current to the motor 1017 (element 1086) to rotate the stator of the motor and the shaft of the motor 1017 in the opposite direction to pull the hammer backwards towards the reverse impact surface 262 of the distal connector 250.

FIG. 11 depicts an example apparatus 1100 such as, for example, a computing device that may be communicatively coupled with an orthopedic surgical instrument or impactor such as, orthopedic impactor 100 shown in connection with FIGS. 1-8, in accordance with one or more features of the present disclosure. The apparatus 1100 may be a computer in the form of a smart phone, a tablet, a notebook, a desktop computer, a workstation, or a server. The apparatus 1100 can be combined with any suitable system, device, and method disclosed herein. The apparatus 1100 can include processor(s) 1110, a non-transitory storage medium 1120, communication interface 1130, and a display 1135. The processor(s) 1110 may include one or more processors, such as a programmable processor (e.g., a central processing unit (CPU)). The processor(s) 1110 may include processing circuitry to implement impactor logic circuitry 1115 such as the impactor logic circuitry 1012 shown in FIG. 10A.

The processor(s) 1110 may include memory such as flash memory to contain program code for execution by the processor(s) 1110. In some examples, the processor(s) 1110 may have random access memory to contain a copy of code from flash memory or read only memory to facilitate faster execution of code. In some examples, the processor(s) 1110 may include cache to contain data for faster calculations or execution. In the present example, the processor(s) 1110 include impactor logic circuitry 1115, which includes a user interface manager 1117. The user interface manager 1117 may function as a state machine controlled by keypad inputs, internal events or alarms, boundary conditions, exceptions and supervisory input to the user interface manager 1117. The user interface manager 1117 may process button presses and may update a main screen on the display 1135 reflecting the state of the application.

Upon startup of the user interface manager 1117, a handler may be installed to detect the motor controller 1016 of the orthopedic impactor 1000 and to establish communication with the motor controller 1016. In some examples, the button presses of button(s) 1052 and edit events may be posted to a panel in the display 1135 and may be processed in real-time. Motor controller commands may be executed upon the user's actions via button presses, system states, and error conditions. Furthermore, the user interface manager 1117 may implement alerts, warnings, and notifications and display the alerts, warnings, and notifications via the display 1135. The user interface manager 1117 may also include code to handle the user's response to alerts, warnings, and notifications.

The processor(s) 1110 may operatively couple with a non-transitory storage medium 1120. The non-transitory storage medium 1120 may store logic, code, and/or program instructions executable by the processor(s) 1110 for performing one or more instructions including the impactor logic circuitry 1125. The non-transitory storage medium 1120 may include one or more memory units (e.g., fixed and/or removable media or external storage such as electrically erasable programmable read only memory (EEPROM), a secure digital (SD) card, random-access memory (RAM), a flash drive, solid-state drive, a hard drive, and/or the like). The memory units of the non-transitory storage medium 1120 can store logic, code and/or program instructions executable by the processor(s) 1110 to perform any suitable method described herein. For example, the processor(s) 1110 may execute instructions such as instructions of impactor logic circuitry 1125 causing one or more processors of the processor(s) 1110 to communicate user commands to an orthopedic impactor 1000 (e.g., orthopedic impactor 100 shown in connection with FIGS. 1-8) and/or to communicate events, alerts, operation parameters for the orthopedic impactor 1000, and configurations.

The impactor logic circuitry 1125 may include operation code 1127, panels 1128, and a configuration file 1129. The operation code 1127 may include functionality to set energy boundaries for operation of the orthopedic impactor 1000, set frequency boundaries for operation of the orthopedic impactor 1000, set an operating energy, set an operating frequency, set a hammer detection profile, set a boundary for a push current interrupt count, set a boundary for a pull current interrupt count, set a delay time or dead time interrupt count to establish a frequency of impact, set an operating mode (full swing, flutter, or oscillation), and/or the like.

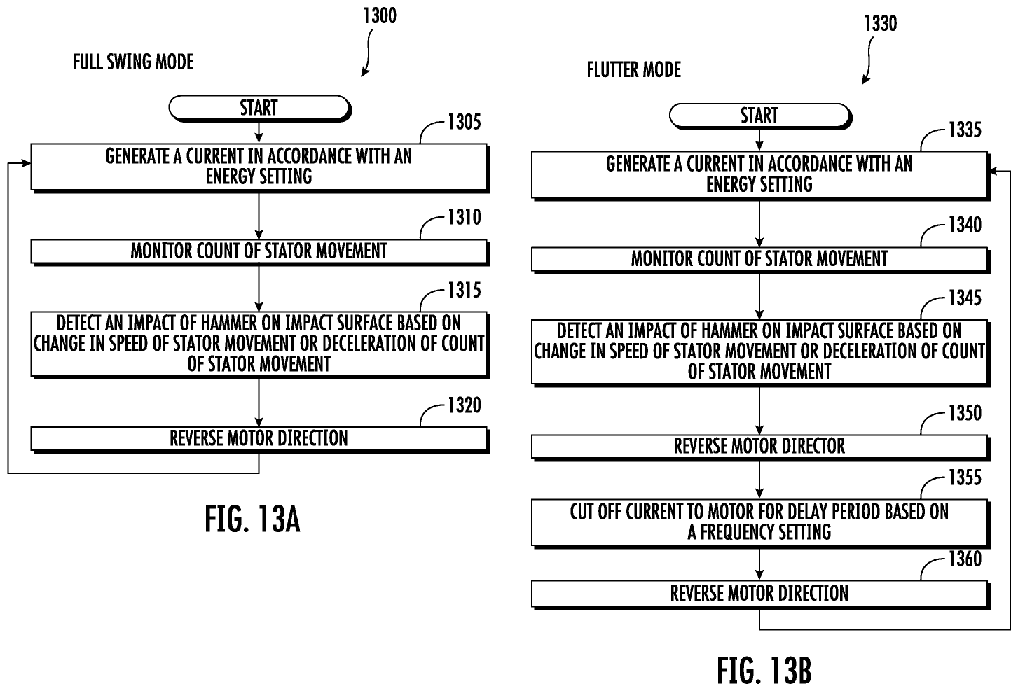

The panels 1128 may define graphical user interfaces for display of information and for receiving input parameters or configurations from a user such as the panels shown in FIGS. 12A-12C. The configuration file 1129 may include user selected parameters such as a motor controller with which to communicate, boundaries for energy (current), boundaries for frequency of impact, numbers of interrupts expected for push current and for pull current, and/or number of interrupts to receive to establish a frequency of impact.

The processor(s) 1110 may couple to a communication interface 1130 to transmit the data, code, or commands to, and/or receive data, code, or commands from, one or more external devices (e.g., a terminal, display device, a smart phone, a tablet, a server, or other remote device). The communication interface 1130 includes circuitry to transmit and receive communications through a wired and/or wireless media such as an Ethernet interface, a wireless fidelity (Wi-Fi) interface, a Bluetooth interface such as a Bluetooth Low Energy (BLE) interface, a cellular data interface, and/or the like. In some examples, the communication interface 1130 may implement logic such as code in a baseband processor to interact with a radio and front end module coupled with an antenna (physical layer device) to transmit and receive wireless communications to/from the orthopedic impactor 1000. For example, the communication interface 1130 may implement one or more of local area networks (LAN), wide area networks (WAN), infrared, radio, Bluetooth, Wi-Fi, point-to-point (P2P) networks, telecommunication networks, cloud communication, and the like.

The processor(s) 1110 may couple to a display 1135 to present panels 1128 as a user interface and/or present other user interface items such as a message or notification via, graphics, video, text, and/or the like. In some examples, the display 1135 may include a display on a terminal, a monitor, another type of display device, a smart phone, a tablet, a server, or a remote device.

FIGS. 12A-12C illustrate example graphical displays of panels for a computing device to monitor and/or control an orthopedic surgical instrument or impactor in accordance with one or more features of the present disclosure. FIG. 12A illustrates a startup panel 1200. The startup panel 1200 may include a graphical button, open device button 1210, to depress via a user input device to identify an attached motor controller such as the motor controller 1016 shown in FIG. 10A. Other examples may include other ways for a user to identify a motor controller such as a pull-down menu, a graphical representation of an attached motor controller, a list of attached motor controllers, and/or the like.

FIG. 12B illustrates an open device panel 1230. The open device panel 1230 may include fields with information about the motor controller as well as connection information about the link to the controller. The open device panel 1230 may also include an “open” button to select the motor controller and a “cancel” button to cancel the process of initiating communications with the motor controller.

FIG. 12C illustrates a control panel 1240 that is connected to a motor controller such as the motor controller 1016 shown in FIG. 10A. The control panel 1240 may include a graphical disable device button 1242 to sever the connection between the motor controller of orthopedic impactor 1000 and the apparatus 1100. The control panel 1240 may include fields with information about the motor controller such as a hammer impact detection profile 1243. In some examples, the user may select the hammer impact detection profile 1243 and adjust parameters such as a threshold velocity at impact, profile of acceleration, a profile of deceleration, a target position for the impact, and/or the like. The profile of acceleration and profile of deceleration may represent thresholds for detection of an impact.

The control panel 1240 may include a current mode to describe the energy to apply to the motor 1017 to drive the hammer 210. For instance, the control panel 1240 includes a push current 1244 setting of 2800 milliamperes (mA), a pull current 1245 setting of 2800 mA, a number (20) of push interrupts 1246, a number (20) of pull interrupts 1248, and a number (0) of dead (delay time) interrupts 1250 to set a frequency of impact.

In the present example, the control panel 1240 also includes an operation mode selector 1254 that allows a user to set the operation mode (full swing, flutter, oscillation) of the orthopedic impactor 1000 via the apparatus 1100.

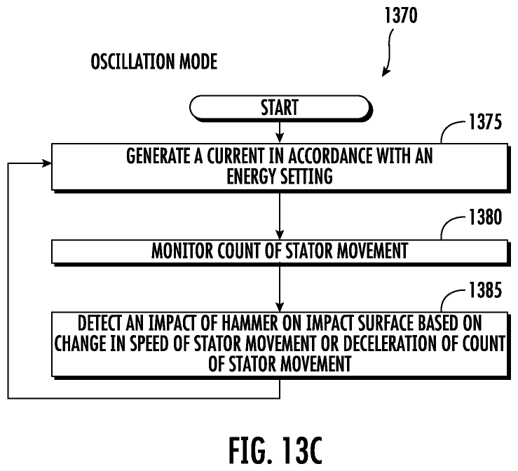

FIGS. 13A-13C illustrate example flowcharts of motor operation in different modes of operation for an orthopedic surgical instrument or impactor in accordance with one or more features of the present disclosure. FIG. 13A illustrates an example flowchart 1300 for full swing mode operation of the impactor such as the orthopedic impactor 1000 shown in FIG. 10A. The flowchart 1300 begins with generating a current in accordance with an energy setting (element 1305). In many examples, the user interface manager may allow a user to select a current in amperes, milliamperes, or the like to set the energy of impact of a hammer 210 of the impactor. The current provides an electromotive force to rotate a motor stator, the motor shaft, a swashplate, and a wobble shaft to accelerate the hammer towards an impact surface.

The motor such as the motor 110 shown in FIG. 3 may include an encoder coupled with the shaft of the motor to provide a count representing incremental rotations of a stator of the motor. For instance, a motor with a 14:1 gear ratio may output 2000 counts per full rotation of the stator of the motor. Some examples monitor the count of the stator movement (element 1310) to determine, e.g., the velocity of the stator, the acceleration of the stator, the deceleration of the stator, and/or the like. In some examples, based on the count of the stator movement, the impact logic circuitry may determine when a change in the count, the velocity, the acceleration, the deceleration, and/or the like may represent an impact of the hammer 210 on an impact surface of a distal connector (element 1315) such as the forward impact surface 260 or the reverse impact surface 262 of the distal connector 250.

After detecting the impact, the impactor logic circuitry may generate a reverse current to reverse the direction of the motor (element 1320). In some examples, prior to applying the reverse current, the impactor logic circuitry may wait for a delay time or a dead time. In some examples, the delay time or dead time may be a count of interrupts associated with clock cycles of a clock circuit in the impactor such as a clock circuit of the processor(s) 1010 of the orthopedic impactor 1000 shown in FIG. 10A.

FIG. 13B illustrates an example flowchart 1330 for flutter mode operation of the impactor such as the orthopedic impactor 1000 shown in FIG. 10A. The flowchart 1330 may begin with generation of a current in accordance with an energy setting (element 1335). For instance, a user of the impactor may select an energy setting by pressing an up button or a down button (plus button or minus button) to set the energy at the level the user desires and the impactor may apply a current associated with that energy setting to the motor of the impactor.

At element 1340, the impactor logic circuitry may monitor a count of stator movements and, at element 1345, the impactor logic circuitry may detect an impact of hammer on impact surface based on change in speed of stator movement or deceleration of count of stator movement.

After detecting the impact, the impactor logic circuitry may reverse the motor direction (element 1350) to move the hammer into a position away from the impact surface and cut off current to the motor for a delay period or dead time based on a frequency setting selected by the user (element 1355). For instance, the user may use plus or minus buttons (or up or down buttons) associated with the frequency setting for the impacts of the hammer on an impact surface. The impact logic circuitry may incorporate a delay via the dead time or delay time period to set the frequency of impact to the user selected frequency of impact. In some examples, the dead time or delay time period may represent clock cycle counts determined by the impact logic circuitry via counts of interrupts associated with the clock cycles.

After the expiration of the dead time or delay time period, the impact logic circuitry may reverse the direction of the motor (element 1360) and generate the current at element 1335.

FIG. 13C illustrates an example flowchart 1370 for oscillation mode operation of the orthopedic impactor such as the orthopedic impactor 1000 shown in FIG. 10A. The flowchart 1370 may begin with the impact logic circuitry generating a current based on the energy setting selected by a user (element 1375). The impact logic circuitry may monitor the stator movement count (element 1380) after application of the current to detect an impact of hammer on impact surface based on change in speed of stator movement or deceleration of count of stator movement (element 1385). For oscillation mode, some examples may detect the impact to log the event, verify that parameters of the impact are within expected parameters, notify a user if parameters fall outside expected parameters, and/or the like. However, in oscillation mode, the impact logic circuitry may continue to drive the motor in the same direction, allowing the hammer to impact the forward and reverse impact surfaces of the distal connector.

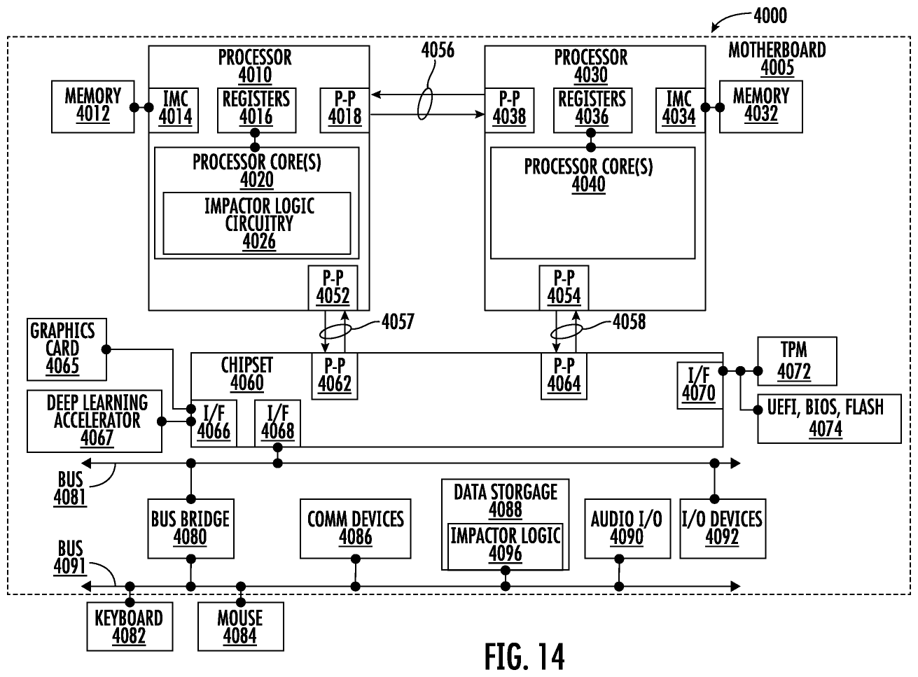

FIG. 14 illustrates another example computing device 4000 communicatively coupled with the orthopedic surgical instrument or impactor in accordance with one or more features of the present disclosure such as the apparatus 1100 shown in FIG. 10B coupled with the orthopedic impactor 1000 shown in FIG. 10A. The computing device 4000 is a computer system with multiple processor cores such as a distributed computing system, supercomputer, high-performance computing system, computing cluster, mainframe computer, mini-computer, client-server system, personal computer (PC), workstation, server, portable computer, laptop computer, tablet computer, handheld device such as a personal digital assistant (PDA), or other device for processing, displaying, or transmitting information. Similar examples may include, e.g., entertainment devices such as a portable music player or a portable video player, a smart phone or other cellular phone, a telephone, a digital video camera, a digital still camera, an external storage device, or the like. Further examples implement larger scale server configurations. In other examples, the computing device 4000 may have a single processor with one core or more than one processor. Note that the term “processor” refers to a processor with a single core or a processor package with multiple processor cores.

As shown in FIG. 14, computing device 4000 includes a motherboard 4005 for mounting platform components. The motherboard 4005 is a point-to-point interconnect platform that includes a first processor 4010 and a second processor 4030 coupled via a point-to-point interconnect 4056 such as an Ultra Path Interconnect (UPI). In other examples, the computing device 4000 may be of another bus architecture, such as a multi-drop bus. Furthermore, each of the first and second processors 4010 and 4030 may be processor packages with multiple processor cores including processor core(s) 4020 and 4040, respectively. While the computing device 4000 is an example of a two-socket (2S) platform, other examples may include more than two sockets or one socket. For example, some examples may include a four-socket (4S) platform or an eight-socket (8S) platform. Each socket is a mount for a processor and may have a socket identifier. Note that the term platform refers to the motherboard 4005 with certain components mounted such as the first processor 4010 and the chipset 4060. Some platforms may include additional components and some platforms may only include sockets to mount the processors and/or the chipset.

The first processor 4010 includes an integrated memory controller (IMC) 4014 and point-to-point (P-P) interconnects 4018 and 4052. Similarly, the second processor 4030 includes an IMC 4034 and P-P interconnects 4038 and 4054. The IMC's 4014 and 4034 couple the first and second processors 4010 and 4030, respectively, to respective memories, a memory 4012 and a memory 4032. The memories 4012 and 4032 may be portions of the main memory (e.g., a dynamic random-access memory (DRAM)) for the platform such as double data rate type 3 (DDR3) or type 4 (DDR4) synchronous DRAM (SDRAM). In the present example, the memories 4012 and 4032 locally attach to the respective first and second processors 4010 and 4030. In other examples, the main memory may couple with the processors via a bus and shared memory hub.

The first and second processors 4010 and 4030 include caches coupled with each of the processor core(s) 4020 and 4040, respectively. In the present example, the processor core(s) 4020 of the first processor 4010 include an impactor logic circuitry 4026 such as the impactor logic circuitry 1012, 1022, 1115, and 1125 shown in FIGS. 10A and 10B. The impactor logic circuitry 4026 may represent circuitry configured to implement the functionality within the processor core(s) 4020 or may represent a combination of the circuitry within a processor and a medium to store all or part of the functionality of the impactor logic circuitry 4026 in memory such as cache, the memory 4012, buffers, registers, and/or the like. In several examples, the functionality of the impactor logic circuitry 4026 resides in whole or in part as code in a memory such as the impactor logic circuitry 4096 in the data storage unit 4088 attached to the first processor 4010 via a chipset 4060 such as the impactor logic circuitry 1125 shown in FIG. 10B. The functionality of the impactor logic circuitry 4026 may also reside in whole or in part in memory such as the memory 4012 and/or a cache of the processor. Furthermore, the functionality of the impactor logic circuitry 4026 may also reside in whole or in part as circuitry within the first processor 4010 and may perform operations, e.g., within registers or buffers such as the registers 4016 within the first processor 4010, or within an instruction pipeline of the first processor 4010.

In other examples, more than one of the first and second processors 4010 and 4030 may include functionality of the impactor logic circuitry 4026 such as the second processor 4030 and/or the processor within the deep learning accelerator 4067 coupled with the chipset 4060 via an interface (I/F) 4066. The I/F 4066 may be, for example, a Peripheral Component Interconnect-enhanced (PCI-e).

The first processor 4010 couples to a chipset 4060 via P-P interconnects 4052 and 4062 and the second processor 4030 couples to a chipset 4060 via P-P interconnects 4054 and 4064. Direct Media Interfaces (DMIs) 4057 and 4058 may couple the P-P interconnects 4052 and 4062 and the P-P interconnects 4054 and 4064, respectively. The DMI may be a high-speed interconnect that facilitates, e.g., eight Giga Transfers per second (GT/s) such as DMI 3.0. In other examples, the first and second processors 4010 and 4030 may interconnect via a bus.

The chipset 4060 may include a controller hub such as a platform controller hub (PCH). The chipset 4060 may include a system clock to perform clocking functions and include interfaces for an I/O bus such as a universal serial bus (USB), peripheral component interconnects (PCIs), serial peripheral interconnects (SPIs), integrated interconnects (I2Cs), and the like, to facilitate connection of peripheral devices on the platform. In other examples, the chipset 4060 may include more than one controller hub such as a chipset with a memory controller hub, a graphics controller hub, and an input/output (I/O) controller hub.

In the present example, the chipset 4060 couples with a trusted platform module (TPM) 4072 and the unified extensible firmware interface (UEFI), Basic Input-Output System (BIOS), Flash component 4074 via an interface (I/F) 4070. The TPM 4072 is a dedicated microcontroller designed to secure hardware by integrating cryptographic keys into devices. The UEFI, BIOS, Flash component 4074 may provide pre-boot code.

Furthermore, chipset 4060 includes an I/F 4066 to couple chipset 4060 with a high-performance graphics engine, graphics card 4065. In other examples, the computing device 4000 may include a flexible display interface (FDI) between the first and second processors 4010 and 4030 and the chipset 4060. The FDI interconnects a graphics processor core in a processor with the chipset 4060.

Various I/O devices 4092 couple to the bus 4081, along with a bus bridge 4080 which couples the bus 4081 to a second bus 4091 and an I/F 4068 that connects the bus 4081 with the chipset 4060. In some examples, the second bus 4091 may be a low pin count (LPC) bus. Various devices may couple to the second bus 4091 including, for example, a keyboard 4082, a mouse 4084, communication devices 4086 and a data storage unit 4088 that may store code such as the impactor logic circuitry 4096. Furthermore, an audio I/O 4090 may couple to second bus 4091. Many of the I/O devices 4092, communication devices 4086, and the data storage unit 4088 may reside on the motherboard 4005 while the keyboard 4082 and the mouse 4084 may be add-on peripherals. In other examples, some or all the I/O devices 4092, communication devices 4086, and the data storage unit 4088 are add-on peripherals and do not reside on the motherboard 4005.

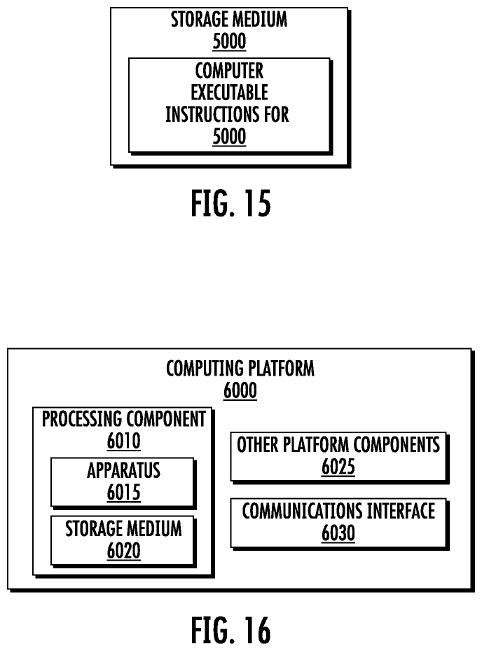

FIGS. 15 and 16 illustrate example storage mediums and computing platforms for an orthopedic surgical instrument or impactor in accordance with one or more features of the present disclosure. FIG. 15 illustrates an example of a storage medium 5000 to store impactor logic such as the impactor logic circuitry 1012, 1022, 1115, and 1125 illustrated in FIGS. 10A and 10B. Storage medium 5000 may include an article of manufacture. In some examples, storage medium 5000 may include any non-transitory computer readable medium or machine readable medium, such as an optical, magnetic or semiconductor storage. Storage medium 5000 may store various types of computer executable instructions, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 16 illustrates an example computing platform 6000. In some examples, as shown in FIG. 16, computing platform 6000 may include a processing component 6010, other platform components or a communications interface 6030. According to some examples, computing platform 6000 may be implemented in a computing device such as a server in a system such as a data center or server farm that supports a manager or controller for managing configurable computing resources as mentioned above. Furthermore, the communications interface 6030 may include a wake-up radio (WUR) and may be capable of waking up a main radio of the computing platform 6000.

According to some examples, processing component 6010 may execute processing operations or logic for apparatus 6015 described herein such as the impactor logic circuitry 1012, 1022, 1115, and 1125 illustrated in FIGS. 10A and 10B. Processing component 6010 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 6020, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 6025 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 6030 may include logic and/or features to support a communication interface. For these examples, communications interface 6030 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 6000 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 6000 described herein, may be included or omitted in various examples of computing platform 6000, as suitably desired.

The components and features of computing platform 6000 may be implemented using any combination of discrete circuitry, ASICs, logic gates and/or single chip architectures. Further, the features of computing platform 6000 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the exemplary computing platform 6000 shown in the block diagram of FIG. 16 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in examples.

One or more aspects of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

The foregoing description has broad application. While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples. Rather these examples should be considered as illustrative and not restrictive in character. All changes and modifications that come within the spirit of the invention are to be considered within the scope of the disclosure. The present disclosure should be given the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counter-clockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular examples. Such terms are not generally limiting to the scope of the claims made herein. Any example or feature of any section, portion, or any other component shown or particularly described in relation to various examples of similar sections, portions, or components herein may be interchangeably applied to any other similar example or feature shown or described herein.

It should be understood that, as described herein, an “embodiment” or an “example” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated embodiments or examples include any other manners of embodying the described concepts or features, such as may be understood by one of ordinary skill in the art upon learning the concepts or features from the present disclosure, and are within the scope of the disclosure. Furthermore, references to an “embodiment” or an “example” of the present disclosure are not intended to be interpreted as excluding the existence of additional embodiments or examples that also incorporate the recited features.

In addition, it will be appreciated that while the Figures may show one or more examples of concepts or features together in a single example of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one example can be used separately, or with another example to yield a still further example. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples or configurations. Moreover, the following claims are hereby incorporated into this Detailed Description by this reference, with each claim standing on its own as a separate example of the present disclosure.

1. An orthopedic surgical instrument arranged and configured to transmit a forward and a reverse energy, the orthopedic surgical impactor comprising:
a housing;
a motor positioned within the housing;
a power supply configured to supply power to the motor;
a trigger assembly configured to activate the motor; and
a mechanical mechanism for converting rotary motion from the motor to reciprocating linear motion of a sliding hammer so that the sliding hammer contacts an impact surface, the mechanical mechanism including a swashplate and a wobble shaft.

2. The orthopedic surgical impactor of claim 1, wherein the motor includes an output shaft, the output shaft is coupled to the wobble shaft, the wobble shaft is coupled to the swashplate, the swashplate is coupled to the sliding hammer so that, in use, rotation of the motor rotates the output shaft, which rotates the wobble shaft, which converts the rotation into linear motion of the sliding hammer via interaction of the wobble shaft and the swashplate.

3. The orthopedic surgical impactor of claim 2, wherein the wobble shaft includes a first end and a second end, the first end is coupled to the output shaft of the motor, the second end is coupled to the swashplate, the second end of the wobble shaft is angled relative to the first end of the wobble shaft.

4. The orthopedic surgical impactor of claim 3, wherein the first end of the wobble shaft includes an internal cavity arranged and configured to receive the output shaft of the motor.

5. The orthopedic surgical impactor of claim 3, wherein the swashplate includes an internal bore arranged and configured to receive the second end of the wobble shaft.

6. The orthopedic surgical impactor of claim 5, wherein the swashplate include an intermediate ball bearing assembly disposed between an inner surface of the internal bore of the swashplate and an outer surface of the second end of the wobble shaft.

7. The orthopedic surgical impactor of claim 3, wherein the swashplate includes a leg extending therefrom, the leg being operatively coupled to the sliding hammer.

8. The orthopedic surgical impactor of claim 7, wherein the sliding hammer is part of an internal hammer assembly including the sliding hammer, an impact mechanism housing, and a coupling mechanism.

9. The orthopedic surgical impactor of claim 8, wherein the impact mechanism housing is coupled to an end of the motor.

10. The orthopedic surgical impactor of claim 8, wherein the impact mechanism housing includes a longitudinal slot formed in an outer surface thereof, the longitudinal slot arranged and configured to enable the leg of the swashplate to extend therethrough so that the leg can operatively engage the sliding hammer via the coupling mechanism.

11. The orthopedic surgical impactor of claim 10, wherein the coupling mechanism is a plunger and a pin.

12. The orthopedic surgical impactor of claim 1, further comprising a distal connector including a coupling mechanism including an internal cavity arranged and configured to receive one of a shaft of an adapter, a surgical tool, or an orthopedic implant, the coupling mechanism further including a pair of spring-loaded fingers to engage the shaft of the adapter, the surgical tool, or the orthopedic implant.

13. The orthopedic surgical impactor of claim 1, further comprising a control mechanism arranged and configured to adjust one of the forward and reverse energy, frequency, and a combination thereof.

14. The orthopedic surgical impactor of claim 13, wherein the control mechanism includes multiple modes of operation to enable a user to vary the forward and reverse energy, frequency, and a combination thereof.

15. The orthopedic surgical impactor of claim 14, wherein the multiple modes of operation include a full swing mode arranged and configured to provide maximum forward and reverse energy, a flutter mode arranged and configured to enable the sliding hammer to oscillate with variable frequencies or amplitudes, an oscillation mode arranged and configured to operate the motor in one direction to impact both forward and reverse impact surfaces, and a combination thereof.
